(19) United States
(12) Patent Application Publication
(10) Pub. No.: US 2025/0032307 A1
(43) Pub. Date: Jan. 30, 2025

(54) HEMP-BASED MOUTHGUARD

(71) Applicants: Cheryl SMITH, Tarzana, CA (US); Mote LONDON, Tarzana, CA (US)

(72) Inventors: Cheryl SMITH, Tarzana, CA (US); Mote LONDON, Tarzana, CA (US)

(21) Appl. No.: 18/361,256

(22) Filed: Jul. 28, 2023

(51) Int. Cl. A61F 5/56; A61L 31/00

(52) U.S. Cl. CPC A61F 5/566; A61L 31/005; A61F 2005/563

(57) ABSTRACT

A hemp-based mouthguard includes first and second hemp-based tabs and a string connector connecting the first and second hemp-based tabs. In one embodiment, a cotton-based string attaches the string connector to the first and second hemp-based tabs. The length of the first and second hemp-based tabs each span at least a portion of the corresponding first side or the second side of a wearer's mouth and are user-adjustable. The string connector has a length sufficient to span the desired spacing between the first and second hemp-based tabs being positioned within the wearer's mouth.

### BACKGROUND

Mouthguards are dental devices designed to prevent teeth grinding during sleep, a condition known as bruxism. In some approaches, they are made of a durable, flexible material, such as silicone or thermoplastic, which can be easily molded to fit over the teeth and gums. Mouthguards serve as a cushioning barrier between the upper and lower teeth. The use of mouthguards has become increasingly popular as awareness grows about the importance of protecting the oral cavity from potential damage and reducing the likelihood of dental emergencies.

### BRIEF SUMMARY

Implementations of the present disclosure relate to a mouthguard having first and second hemp-based tabs and a string connector connecting the tabs.

### BRIEF DESCRIPTION OF THE DRAWINGS

In order to describe the manner in which the present disclosure can be implemented, a more particular description will be rendered by reference to specific implementations thereof which are illustrated in the appended drawings. For better understanding, the like elements have been designated by like reference numbers throughout the various accompanying figures. Understanding that these drawings depict only typical implementations of the invention and are not therefore to be considered to be limiting of its scope, the invention will be described and explained with additional specificity and detail through the use of the accompanying drawings in which:

FIG. 1 is a front view of a hemp-based mouthguard, according to an implementation of the present disclosure, suitable to be placed between the upper and lower teeth of a wearer's mouth.

FIG. 2 is a rear view of the hemp-based mouthguard of FIG. 1.

FIG. 3 is a side perspective view of the hemp-based mouthguard of FIG. 1.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

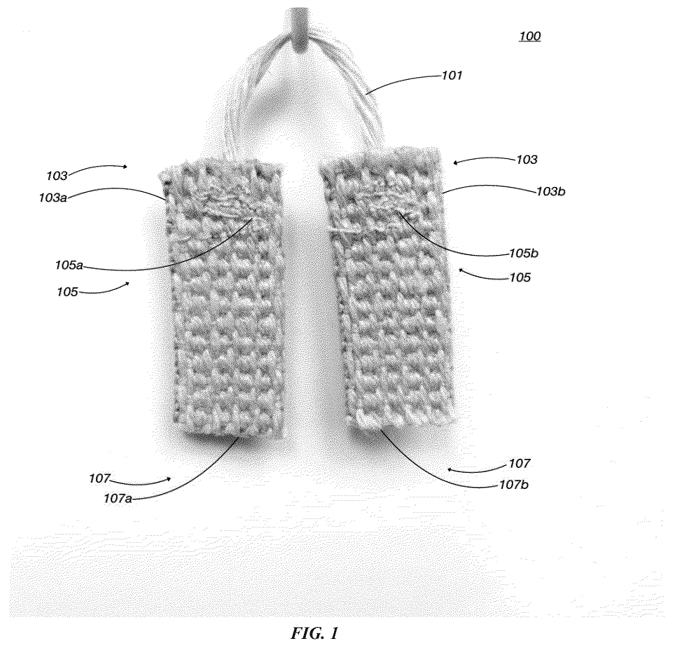

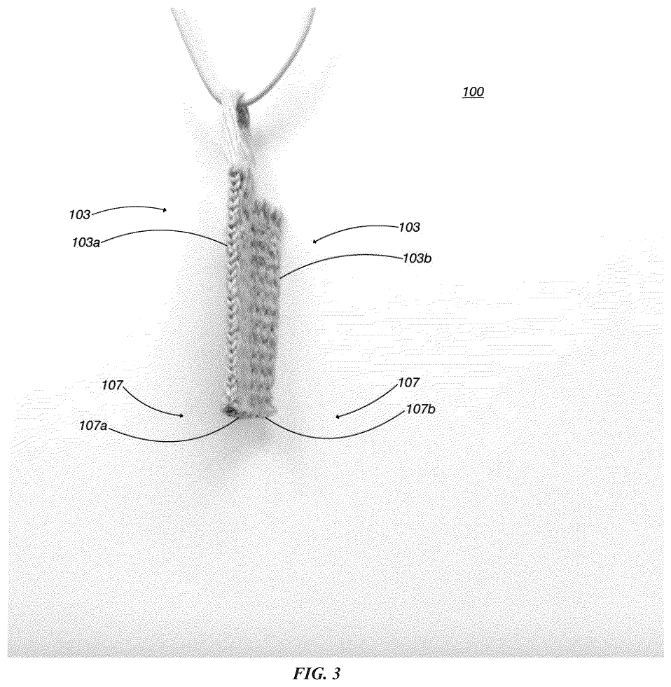

In this disclosure, as in FIGS. 1-3, specific terminology is employed for the sake of clarity. The invention as claimed in this application, however, is not intended to be limited to specific terminology so selected, and it is to be understood that each specific element includes all technical equivalents that operate in a similar manner to accomplish similar functions.

One or more embodiment in accordance with the present invention generally relates to mouthguards and more particularly a teeth-separating system made from hemp that provides a physical barrier between the wearer's top and bottom teeth to reduce teeth grinding.

Reference will now be made to the figures to describe various aspects of example embodiments of the disclosure. FIG. 1 is a front view of a mouthguard 100, which is comprised of a plurality of components. Such components in at least one context include a first tab 103a, a second tab 103b, and a string connector 101. First and second tabs 103a/103b are also referred to using tabs 103. Such components are individually configured and correlated with respect to each other so as to attain the desired mouthguard 100. The mouthguard 100 provides a physical barrier between the wearer's upper and lower teeth, which absorbs the forces of grinding and clenching and protects the teeth from damage.

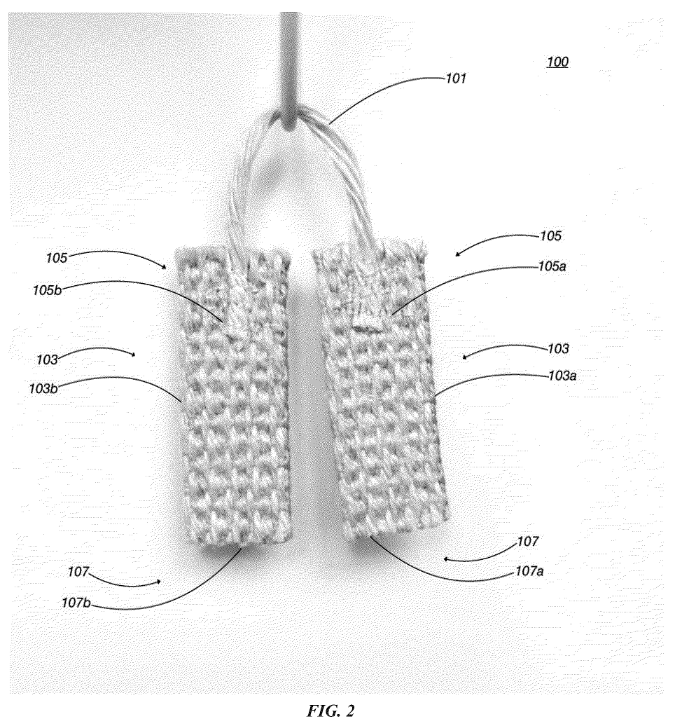

FIG. 1 depicts the mouthguard 100 comprising the first and second tabs 103 (103a, 103b) and the string connector 101. The mouthguard 100 is positioned in a generally horizontal plane when in use between the teeth of a wearer while the wearer is in an upright position. The top surfaces of the first and second tabs 103, as in FIG. 1, are positioned to be in contact with the wearer's upper teeth. The bottom surfaces of the first and second tabs 103, as in FIG. 2, are positioned to be in contact with the wearer's lower teeth. The first and second tabs 103 are connected to each other with the string connector 101. The string connector 101 is attached to the first and second tabs 103 using a string 105 (individual strings 105a, 105b are used for attaching the string connector 101 to the corresponding first and second tabs 103). In FIG. 2, in some embodiments, the string connector 101 is sewn to either the bottom or the top of the first and second tabs 103 using the string 105 to attach the first and second tabs 103 to the string connector 101. In one or more embodiments, the string 105 is a cotton-based string. In other embodiments, the string 105 is made from 100% organic cotton.

In FIGS. 1, 2, and 3, the first and second tabs 103 are user adjustable and are shaped to receive one or more teeth of a wearer. The first and second tabs 103 are cut to a length that is in excess and may be additionally trimmed and shortened according to the length desired by the wearer. In some embodiments, the user may cut one or more corners of the tabs 103 to fit the wearer's mouth without affecting the durability of the mouthguard 100. In some embodiments, the user may cut the edges 107 (107a, 107b) using scissors to shorten the length of the tabs according to the wearer's desired length. Trimming the edges 107 of the first and second tabs 103 may result in frayed or unfinished edges. In one embodiment, the trimmed edges 107 may be sealed to provide a finished edge or completed appearance by using a woven material for the tabs 103. In an embodiment, the woven material is a closed weave pattern. The edges 107 (107a, 107b) of the first and second tabs 103, in FIGS. 1, 2, and 3, are trimmed to have a raw edge.

In at least one embodiment, the first and second tabs 103 have an approximate width ranging from 1.5 centimeters to 0.95 centimeters along the length of the tabs 103. In some embodiments, the width may be narrower or wider.

In some embodiments, the length of the first and second tabs 103 may vary according to the wearer's preferences and span at least a portion of the corresponding first and second side of the wearer's mouth with an approximate length ranging from 2.0 centimeters to 4.0 centimeters.

In some embodiments, the thickness of the first and second tabs 103 may vary depending on the fabric. In some embodiments, the thickness of the first and second tabs 103 ranges from 0.25 cm-0.75 cm. In some embodiments, the thickness of the first and second tabs 103 is approximately 0.25 cm. In some embodiments, the thickness may be narrower or thicker. In some embodiments, the thickness of the first and second tabs 103 is approximately 0.75 cm. The thickness varies in order to accommodate the mouth of the user. In some embodiments, a thicker thickness is achieved by doubling or tripling the fabric of the first and second tabs 103 and sewing the fabrics together either with the same string 105 or a separate string of similar composition.

In some embodiments, the first and second tabs 103 are made from a fabric consisting of long fibers that are woven, crocheted, knitted, or another fabric pattern that provides flexibility and durability to the mouthguard 100 when the first and second tabs 103 are under the stress caused by the clenching and grinding between the wearer's teeth. In at least one embodiment, woven fabrics, interlaced to have a distinct pattern and texture, are preferred over other fabrics made from compressed and matted fibers, which are less durable and disintegrate more easily. The material for the first and second tabs 103 is chosen from the class of organic materials that have long fibers and are in their most raw and naturally occurring form, such as jute, straw, bamboo, or the like. In other embodiments, the first and second tabs 103 are made from fabrics like silk or linen. Fabrics and materials in this category are selected to ensure that the wearer, including at least the wearer's teeth, gums, saliva, and mouth, are not exposed to harmful chemicals, toxins, or volatile organic compounds (VOCs), that could potentially cause irritations, allergies, or harm to the wearer. These materials and fabrics also provide the benefit of being eco-friendly.

In some embodiments, the first and second tabs 103 are made from organic hemp webbing. Hemp is naturally anti-fungal, anti-microbial, and hypoallergenic. Due to the hemp-based composition of the first and second tabs 103, the first and second tabs 103 are susceptible to impressions from the first and second tabs' 103 surrounding environment when subjected to pressure. However, the first and second tabs 103 can readily withstand the pressures exerted by the wearer's teeth and mouth without reaching the first and second tabs' 103 maximum compressions. This applies irrespective of whether the mouthguard 100 is wet (either after being placed in the wearer's mouth or prior to insertion into the wearer's mouth, i.e., pre-wetted) or dry. This provides the wearer with a mouthguard 100 that is tailored according to the impression left from the wearer's teeth when applying pressure to the first and second tabs 103. As the wearer continues to use the mouthguard 100 and apply pressure from their teeth and mouth to the first and second tabs 103, the mouthguard 100 will continue to become more tailored to the wearer's teeth and mouth. Due to the durable and flexible nature of the hemp webbing, in some embodiments, the mouthguard 100 is constantly adapting to the wearer's teeth because the impressions left on the first and second tabs 103 are pliable and not permanent.

In FIGS. 1, 2, and 3, the string connector 101 connects the first and second tabs 103 to each other and has a length sufficient to span the desired spacing between the first and second tabs 103 when positioned in the wearer's mouth. In certain embodiments, the string connector 101 has a standard length of approximately 4.00 centimeters and can be adjusted based on the width of the wearer's mouth to sit partially or fully extended between the first and second sides of the wearer's mouth. The string connector's 101 flexibility enables the wearer to adjust the first and second tabs 103, angling them towards or away from each other. This adjustment may depend on the width difference between the front and back of the wearer's mouth. In some embodiments, the string connector 101 is made using hemp wick that may be coated in beeswax, which reduces potential fraying and keeps the hemp wick free from bacteria due to the beeswax's anti-microbial properties. The beeswax coating melts off cleanly from the string connector 101 when the mouthguard 100 is sterilized (i.e., via boiling water) prior to the wearer's first use. The combined characteristics of both the first and second hemp-based tabs 103 and the string connector 101 provide the wearer with a range of customization options.

The foregoing description and drawings comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions and the associated drawings. Although specific terms may be employed herein, they are used in a generic and descriptive sense only and not for purposes of limitation. Accordingly, the present invention is not limited to the specific embodiments illustrated herein, but is limited only by the following claims.

We claim:

1. A mouthguard, comprising:
first and second hemp-based tabs, each tab shaped to receive one or more teeth of a
wearer on a corresponding first or second side of the wearer's mouth; and
a string connector connecting the first and second hemp-based tabs.

2. The mouthguard of claim 1, wherein the first and second hemp-based tabs each comprise a hemp webbing with unsealed edges and a length adjustable to the wearer's mouth.

3. The mouthguard of claim 2, wherein the length of the first and second hemp-based tabs are user-adjustable.

4. The mouthguard of claim 1, wherein the string connector is hemp.

5. The mouthguard of claim 1, wherein the string connector is attached to the first and second hemp-based tabs using a cotton-based string.

6. The mouthguard of claim 5, wherein the string connector is sewn to the first and second hemp-based tabs using the cotton-based string.

7. The mouthguard of claim 1, wherein the first and second hemp-based tabs each span at least a portion of the corresponding first side or the second side of the wearer's mouth.

8. The mouthguard of claim 1, wherein the string connector has a length sufficient to span the desired spacing between the first and second hemp-based tabs being positioned within the wearer's mouth.

9. A mouthguard, comprising:
first and second hemp-based tabs, each tab shaped to receive one or more teeth of a
wearer on a corresponding first or second side of the wearer's mouth;
a string connector connecting the first and second hemp-based tabs; and
a cotton-based string attaching the string connector to the first and second hemp-based tabs.

10. The mouthguard of claim 6, wherein the first and second hemp-based tabs each comprise a hemp webbing with unsealed edges and a length adjustable to the wearer's mouth.

11. The mouthguard of claim 7, wherein the length of the first and second hemp-based tabs are user-adjustable.

12. The mouthguard of claim 6, wherein the string connector is hemp.

13. The mouthguard of claim 6, wherein the string connector is sewn to the first and second hemp-based tabs using the cotton-based string.

14. The mouthguard of claim 6, wherein the first and second hemp-based tabs each span at least a portion of the corresponding first side or the second side of the wearer's mouth.

15. The mouthguard of claim 6, wherein the string connector has a length sufficient to span the desired spacing between the first and second hemp-based tabs being positioned within the wearer's mouth.

16. A method of fabricating a mouthguard, comprising:
forming first and second hemp-based tabs from cutting hemp webbing to a length
adjustable to a wearer's mouth; and
connecting the first and second hemp-based tabs with a string connector,
wherein the first and second hemp-based tabs each span at least a portion of the
corresponding first side or the second side of the wearer's mouth, and
wherein the string connector has a length sufficient to span the desired spacing between
the first and second hemp-based tabs being positioned within the wearer's mouth.

17. The method of claim 16, further comprising the step of a user adjusting a length of at least one of the first or second hemp-based tabs.

18. The method of claim 16, further comprising the step of a user adjusting the length of at least one of the first or second hemp-based tabs using scissors to trim edges of the first or second hemp-based tabs.

19. The method of claim 16, further comprising the step of a wearer biting the mouthguard to create an imprint of the wearer's teeth on the first and second hemp-based tabs.

20. The method of claim 19, further comprising the step of the wearer biting the mouthguard a plurality of times.
